(19) United States
(12) Patent Application Publication
(10) Pub. No.: US 2026/0346002 A1
(43) Pub. Date: Nov. 5, 2026

(54) THREADED LEAD FOR IMPLANT AND FIXATION OF ELECTRODE

(71) Applicant: Medtronic, Inc., Minneapolis, MN (US)

(72) Inventors: Samuel I. Owings, Roseville, MN (US); Maggie J. Pistella, Minneapolis, MN (US); Douglas S. Hine, Forest Lake, MN (US); Mark W. Shepler, White Bear Lake, MN (US)

(21) Appl. No.: 19/667,964

(22) Filed: May 5, 2026

Related U.S. Application Data
(60) Provisional application No. 63/800,159, filed on May 5, 2025.

(51) Int. Cl. A61N 1/05

(52) U.S. Cl. CPC A61N 1/0573; A61N 1/0563; A61N 1/0565

(57) ABSTRACT

An implantable medical lead may comprise a lead body extending from a proximal end to a distal end along a longitudinal axis, a first electrode extending distally from the distal end, and a thread formed on a radially outer surface of the lead body proximal of the first electrode, the thread configured to engage tissue of a patient as the first electrode is advanced into the tissue.

### RELATED APPLICATIONS

This application claims the benefit of U.S. Provisional Application Ser. No. 63/800,159, filed May 5, 2025, the entire contents of which are incorporated herein by reference.

### TECHNICAL FIELD

The disclosure relates generally to medical device lead fixation.

### BACKGROUND

Medical devices may be used to monitor physiological signals of a patient. For example, some medical devices are configured to sense cardiac electrogram (EGM) signals indicative of the electrical activity of the heart via electrodes. Some medical devices may be configured to deliver a therapy in conjunction with or separate from the monitoring of physiological signals. For example, cardiac pacing is an effective therapy for treating patients with bradycardia due to sinus node dysfunction or atrioventricular block. Delivery of cardiac pacing to the conduction system of the heart, such as to the Bundle of His or the left bundle branch (LBB), has been proposed to treat such disorders while allowing the heart to contract in a more physiological natural manner.

### SUMMARY

Medical devices like pacemakers or implantable cardioverter-defibrillators (ICDs) may send electrical signals to a heart of a patient. The medical device may deliver pacing to the heart of the patient via a tip electrode, which may be a cathode. The tip electrode may be on a distal tip of a lead or device located in the cardiac tissue (e.g., at a left bundle branch (LBB) of the heart). The distal tip of the lead may be inserted into the cardiac tissue to reduce a capture threshold for the stimulation and to improve anchoring of the lead in the tissue. A return electrode (e.g., an anode electrode) may be located outside the heart, such as at the device's metal case (i.e., the “can”), or more proximally on the lead. An electrical signal sent by the device will travel from the tip electrode in a ventricular septum of the heart, through the heart muscle and associated bundle branches, and then back to the return electrode.

The tip electrode may be configured to deliver conduction system pacing, such as left bundle branch area pacing (LBBAP) therapy. LBBAP is a physiological pacing approach that activates the normal cardiac conduction pathways and provides synchronized contraction of ventricles. LBBAP is associated with improved patient outcomes compared to some therapy modalities such as right ventricular apical pacing (RVA), although patient outcomes vary by patient. Conduction system pacing may involve the placement of the tip electrode (e.g., a distal electrode) of a pacing lead in a heart's electrical conduction system (e.g., in a ventricular septum of the heart) to restore normal electrical conduction and improve cardiac function. A medical device may capture tissue proximate the ventricular septum of the heart using any number of individual electrodes (e.g., two relatively closely spaced electrodes on a lead or pacing device). An electrical signal sent by the device travels from the tip electrode (e.g., the more distal electrode on the lead) in the target cardiac tissue (e.g., the ventricular septum of the heart), through the heart muscle and bundle branches (e.g., LBB), and then back to the anode electrode (e.g., the less distal, i.e., more proximal, electrode on the lead). With this configuration, the tip electrode at the cardiac tissue typically captures the cardiac tissue, and any tissue proximate to or in contact with the return electrode is not captured.

In accordance with techniques of this disclosure, an implantable medical lead may have a thread formed on a distal end of the lead body proximal of the distal tip electrode, e.g., on a spacer between a tip electrode and a return electrode or other more proximal electrode, such as a tip to ring spacer. An implantable medical lead configured with a thread in accordance with the techniques of this disclosure may improve advancement of the lead into cardiac tissue to reach relatively deeper targets, such as the LBB, and may improve fixation of the distal end of the lead into the cardiac tissue. For example, a lead body portion, which may be a spacer between a tip electrode and a return electrode, may have a simple mechanical thread which provides traction to bore into cardiac tissue of the patient. The thread may provide mechanical fixation of the distal end of the lead into the tissue. The thread may improve implantation by providing additional traction during implantation. The thread may resist dislodgement because the thread provides a mechanical advantage against dislodgement. The thread may enable alternative (e.g., non-helical) tip electrode designs and/or alternative spacer designs to improve implant location accuracy, reduce dislodgement, or improve penetration depth accuracy.

In one example, an implantable medical lead comprises: a lead body extending from a proximal end to a distal end along a longitudinal axis; a first electrode extending distally from the distal end; and a thread formed on a radially outer surface of the lead body proximal of the first electrode, the thread configured to engage tissue of a patient as the first electrode is advanced into the tissue.

In another example, a method of implanting a medical device comprises advancing a distal end of an implantable medical lead into a right chamber of the heart, wherein the implantable medical lead comprises: a lead body extending from a proximal end to a distal end along a longitudinal axis; a first electrode extending distally from the distal end; and a thread formed on a radially outer surface of the lead body proximal of the first electrode. The method further comprises rotating of the lead body, wherein the rotating is configured to engage the thread with cardiac tissue of the heart and advancing the lead body via rotation until the first electrode contacts tissue of a heart's conduction system.

In another example, a medical device system comprises an implantable medical lead comprising: a lead body extending from a proximal end to a distal end along a longitudinal axis; a first electrode extending distally from the distal end; and a thread formed on a radially outer surface of the lead body proximal of the first electrode, the thread configured to engage tissue of a patient as the first electrode is advanced into the tissue. The system further comprises an implantable medical device comprising processing circuitry configured to control stimulation circuitry, wherein the stimulation circuitry is configured to deliver cardiac stimulation via the first electrode to tissue of a left ventricle of a patient.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1A is a conceptual diagram that illustrates an example medical system in conjunction with a heart of a patient.

FIG. 1B is a conceptual diagram that illustrates an example medical system including a defibrillation coil in conjunction with a heart of a patient.

FIG. 2 is a conceptual diagram that illustrates a distal portion of an example lead with an example thread.

FIG. 3 is a functional block diagram illustrating an example configuration of an implantable medical device (IMD).

FIG. 4 is a functional block diagram illustrating an example configuration of an external device.

FIGS. 5A, 5B, 5C, 5D, and 5E illustrate example distal electrode configurations.

FIGS. 6A and 6B illustrate example spacers with modified distal ends.

FIG. 7 is a flowchart of an example method for implanting a medical lead in accordance with techniques of this disclosure into a heart of a patient.

### DETAILED DESCRIPTION

A heart relies on a specialized electrical system to generate and transmit electrical signals to coordinate the contraction of its chambers to efficiently pump blood. Abnormalities or disorders of the heart's electrical system may lead to irregular heart rhythms (e.g., arrhythmias) or irregular heart rates (e.g., bradycardia or tachycardia). Medical devices, such as pacemakers, may be used to restore and maintain a normal heart rate and rhythm for patients. Pacemakers may employ conduction system pacing (CSP), (i.e., physiologic pacing), which is a pacing modality to regulate the heartbeat when a heart's natural electrical conduction system is not functioning properly. A cardiac lead of the pacemaker may deliver the CSP to a ventricular septum of the heart. A capture threshold (i.e., the amount of energy before the heart responds) for some forms of CSP, such as left bundle branch (LBB) pacing, may be reduced by increasing a penetration depth of the cardiac lead. In other words, a pacemaker may be able to deliver better LBB pacing to the patient if a cardiac lead is inserted deeper into the ventricular septum. Traditionally, cardiac leads are affixed to the ventricular septum by a wire helix. These wire helices are typically less than 2 millimeters (mm) long. Deeper penetration of the ventricular septum with these helices may be difficult due to the relative width of the wire helix compared to a width of the body of the cardiac lead. Therefore, a cardiac lead which may gently bore into a ventricular septum of a patient and remain fixed for the duration of implantation is desired.

In accordance with techniques of this disclosure, a cardiac lead has a thread on an outer surface of a distal portion of the lead body proximal a distal electrode, e.g., on the tip to ring spacer. The thread may provide additional penetrating traction during implantation of the cardiac lead into tissue. The additional penetrating traction may improve implant accuracy. The thread may define a pitch, where the pitch defines a depth the cardiac lead may advance into tissue per rotation of the cardiac lead. As such, the pitch of the thread may improve an accuracy of implantation depth for the cardiac lead compared to a fixation helix alone because a fixation helix alone only defines a pitch for the longitudinal depth of the fixation helix. In contrast, the thread defines a pitch for the length of the thread, which may be deeper than the fixation helix. The thread may provide additional resistance against dislodgement. Specifically, the thread, which is a simple machine (e.g., a screw) may resist longitudinal movement due to the ridges of the thread. The thread may enable distal electrode design other than a fixation helix. For example, the distal electrode may be an electrode design other than a fixation helix such as a cone, a tapered cone, a dart, a frustum, or any other electrode designs as discussed throughout the disclosure. As such, a thread on a spacer of a cardiac lead may improve cardiac lead placement (e.g., depth and location), improve cardia pacing capture thresholds, reduce cardiac lead dislodgement, and enable alternative distal electrode designs which may thereby improve patient experiences and outcomes as well as improve physician implant experiences.

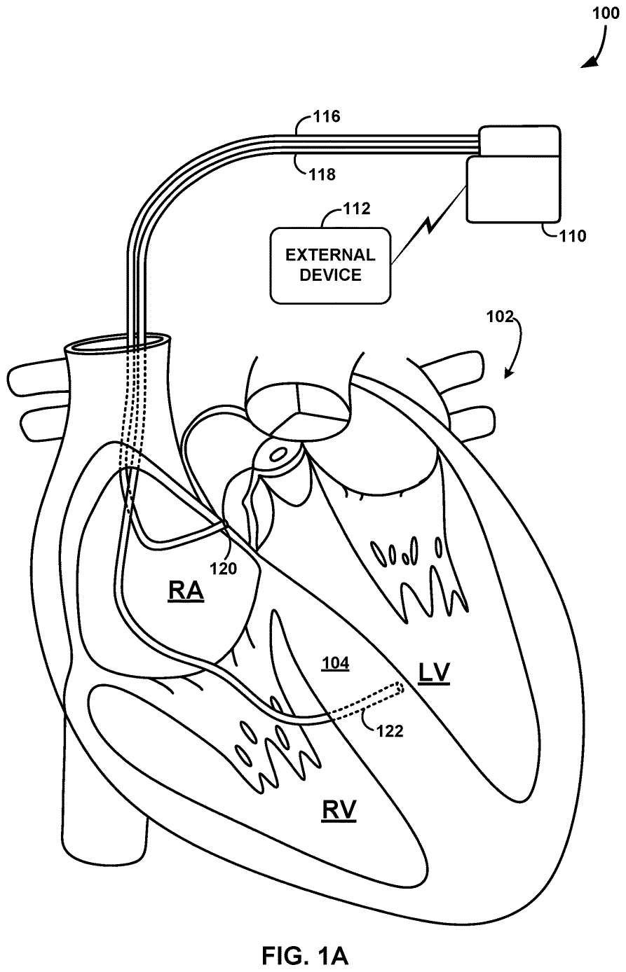

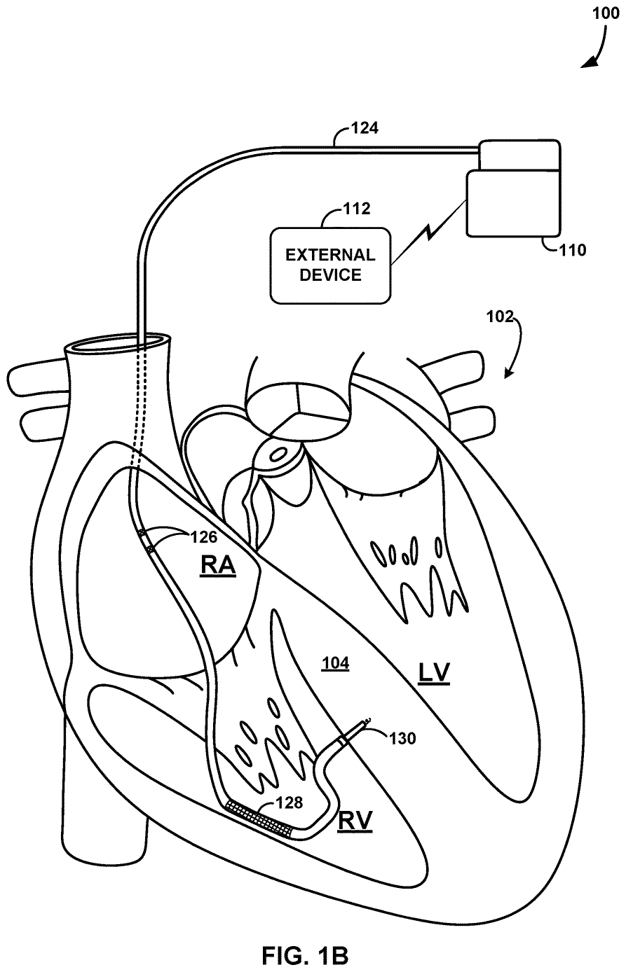

FIG. 1A is a conceptual diagram that illustrates an example medical device system 100 capable of pacing and sensing in a patient's heart 102. FIG. 1B is a conceptual diagram that illustrates an example medical system including a defibrillation coil in conjunction with a heart of a patient. FIGS. 1A and 1B will be discussed together for clarity. Medical device system 100 may include implantable medical device (IMD) 110 and an external device 112. IMD 110 may be in wireless communication with at least one of external device 112 and other devices not pictured in FIG. 1A or 1B. IMD 110 may be implanted within a patient, and coupled to one or more implantable medical leads 116, 118. IMD 110 may be configured to sense one or more cardiac electrograms (EGMs) and deliver conduction system pacing, such as LBBAP, via a plurality of electrodes on leads 116, 118.

External device 112 may be a computing device with a display viewable by the user and an interface for providing input to external device 112 (i.e., a user input mechanism). In some examples, external device 112 may be a notebook computer, tablet computer, workstation, one or more servers, cellular phone, personal digital assistant, or another computing device that may run an application that enables the computing device to interact with IMD 110. In some examples, external device 112 may be implemented as a number of computing systems configured to receive cardiac electrical signals, which may include at least one EGM signal input received directly or indirectly from IMD 110. In some examples, external device 112 is a computing device included in a remote patient monitoring system.

External device 112 is configured to communicate with IMD 110 and, optionally, another computing device (not illustrated in FIG. 1A or 1B), via wireless communication. External device 112, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies).

External device 112 may be used to configure operational parameters for IMD 110. External device 112 may be used to retrieve data from IMD 110. The retrieved data may include values of patient parameters measured by IMD 110, indications of episodes of arrhythmia or other maladies detected by IMD 110, and physiological signals recorded by IMD 110. IMD 110 may be coupled to a heart 102 via transvenous electrical leads 116 and 118. In some examples, IMD 110 may be configured to detect tachyarrhythmias from the sensed cardiac signals and deliver high voltage cardioversion/defibrillation (CV/DF) shocks to heart 102, e.g., for terminating a detected ventricular tachycardia or ventricular fibrillation. Leads 116 and 118 may be inserted into tissue of heart 102 via fixation mechanisms as described herein.

Lead 116 may be a RA lead (“RA lead 116”) and lead 118 may be a ventricular conduction system pacing lead (“VCS pacing lead 118). RA lead 116 and VCS pacing lead 118 may be advanced transvenously to position electrodes for sensing cardiac electrical signals and delivering pacing therapy. RA lead 116 may be positioned such that RA distal end 120 may be implanted at the triangle of Koch. RA distal end 120 may carry pacing and sensing electrodes which may include a tip electrode (not shown) and/or a ring electrode (not shown). Electrodes provide sensing and pacing in the RA and are each connected to a respective insulated conductor extending within the elongated body of RA lead 116 from RA distal end 120 back to IMD 110. IMD 110 may include a connector block configured to receive a lead connector for electrically coupling conductors extending from the distal electrodes to circuitry within a housing via electrical feedthroughs.

VCS lead 118 may be advanced within the right atrium to position at least one electrode including a tip electrode and a ring electrode (not shown) at VCS distal end 122 for pacing and sensing in the vicinity of the VCS, e.g., at or near the LBB, from a right atrial approach, as shown. Such an implantation site may include securing VCS distal end 122 into interventricular septum 104 of heart 102 via one or more fixation mechanisms. Upon implantation, VCS distal end 122 may lie between the bundle of His and the coronary sinus or may be adjacent the tricuspid valve. A tip electrode at VCS distal end 122 may be a helical electrode. In some examples, the tip electrode at VCS distal end 122 may be advanced into the inferior end of the interatrial septum, beneath the AV node and near the tricuspid valve annulus to position tip electrode in, along or proximate to the His bundle. Specifically, electrodes of VCS lead 118 may penetrate through the wall tissue of a first chamber (e.g., the RV in the illustrated example) into septum tissue 104 of heart 102. Penetration of the septal tissue 104 may decrease a stimulation threshold for VCS lead 118. The ring electrode, which may be spaced proximally from tip electrode may be used as the return electrode with the cathode tip electrode for pacing the right and left ventricles via the native His-Purkinje conduction system.

Although RA lead 116 and VCS pacing lead 118 are described as directed to pacing the right atrium and the ventricular conduction system, from the right ventricular side, RA lead 116 and VCS pacing lead 118 may alternatively be placed anywhere within heart 102 such as the left atrium, the left ventricle, the septum 104, through or within any of the valves, and/or through or within any of the arteries or veins connecting to the heart which are suitable for receiving pacing or defibrillation pulses.

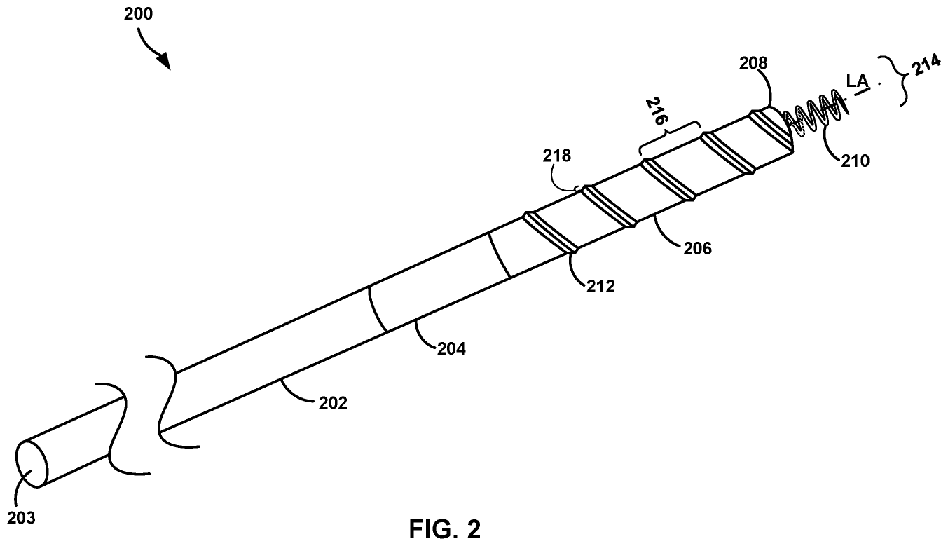

In some examples, distal end of pacing lead 118 may include a fixation feature (not shown in FIG. 1A or 1B) disposed on distal end and radially inward of first electrode. The fixation feature is further described below with respect to FIGS. 2, 5A, 5B, 5C, 5D, 6A, and 6B. The fixation feature may extend distally from VCS distal end 122 deeper into tissue of heart 102 to secure VCS distal end 122. The fixation feature may additionally or alternatively extend radially outwards and proximally from VCS distal end 122. The fixation feature may improve attachment security and/or may improve attachment placement accuracy. The fixation feature may interface with the wall tissue to inhibit unintended movement (e.g., unintended rotation and/or unintended displacement) of VCS lead 118 relative to the wall tissue (e.g., of first electrode within the wall tissue). The fixation feature may define a thread shape. The fixation feature may define one or more of a plurality of shapes as described throughout below. The fixation feature may allow for movement (e.g., rotation) of first electrode in a first direction (e.g., into the wall tissue of heart 102) and inhibit movement of first electrode in a second direction opposite the first direction. Although described primarily herein with respect to lead 118 and lead distal end 122, lead 116 with lead distal end 120 may similarly be configured with a fixation feature as described herein, e.g., to facilitate deeper penetration and fixation of distal end 120 for conduction system and/or left ventricular pacing.

IMD 110 may produce an intracardiac electrogram signal from the cardiac electrical signal received via a sensing electrode vector that may include tip electrode and/or ring electrode of RA lead 116 and/or VCS lead 118. The electrodes and are coupled to respective insulated conductors extending within the elongated body of VCS lead, which provide electrical connection to a proximal lead connector coupled to circuitry of IMD 110 within housing via electrical feedthroughs crossing housing.

A housing of IMD 110 may function as a return electrode for unipolar sensing or pacing configurations with any of the electrodes carried by leads 116 and 118. Electrodes may be used in a bipolar pacing electrode pair for delivering VCS pacing pulses and for receiving a cardiac electrical signal for sensing intrinsic and pacing evoked QRS waveforms. In some examples, IMD 110 may be configured to sense a far field (FF) cardiac signal, e.g., using electrode and housing or using the electrodes and the housing, and/or a near field (NF) cardiac signal, e.g., using the electrodes and, for processing and analysis for determining a capture type. The electrodes may be used in a sensing electrode vector for sensing intrinsic R-waves for use in determining a heart rhythm and a need for electrical stimulation therapy.

In FIG. 1A, IMD 110 is a dual chamber device configured to receive RA lead 116, positioned in the right atrial chamber for delivering atrial pacing pulses and sensing atrial electrical signals via electrodes and. IMD 110 may be configured to sense intrinsic atrial P-waves and deliver atrial pacing pulses in the absence of sensed P-waves. IMD 110 may be configured to deliver atrial synchronized ventricular pacing by setting an AV delay in response to each sensed P-wave or delivered atrial pacing pulse and deliver a ventricular pacing pulse to the VCS via lead 118 upon the expiration of the AV delay.

VCS lead 118 may be advanced transvenously into the RV via the RA for positioning a tip electrode within inter-ventricular septum 104. The tip electrode may be selected as a pacing cathode electrode in combination with ring electrode as the return anode electrode for pacing and capturing the LBB and/or RBB in various examples. In some examples, the pacing magnitude (e.g., pulse amplitude and/or pulse width) may be selected to achieve cathodal capture at the cathode electrode for capturing at least one bundle branch. In some examples, the pacing pulse amplitude and/or pulse width may be selected to achieve cathodal and anodal capture, which may capture the LBB and the right side of the ventricular septum. In some examples, the LBB and the RBB may be captured concurrently to provide dual or bilateral bundle branch (BB) pacing using a single bipolar electrode pair.

In other examples, either a tip electrode or a ring electrode may be selected as a cathode electrode paired with a housing of IMD 110 in a unipolar pacing electrode vector. Unipolar pacing may capture a single BB or location within the ventricles. VCS lead 118 may carry one pacing and one sensing electrode pair, it is to be understood that in other examples, VCS lead 118 may include multiple electrodes along its distal portion to provide one or more selectable bipolar pacing electrode vectors and/or one or more unipolar pacing electrode vectors (e.g., with housing) for delivering pacing pulses to the left and right side of the ventricular septum.

VCS lead 118 may include one or more coil electrodes when IMD 110 is configured as an ICD capable of delivering high voltage shock therapies. The coil electrode may be used in sensing electrode vectors, e.g., with either of the tip electrode or the ring electrode, for sensing a ventricular EGM signal that may be analyzed processing of system (e.g., of IMD 110) for classifying VCS pacing pulse capture type.

As described above, IMD 110 may communicate via wireless telemetry with external device 112. External device 112 may receive EGM signals from IMD 110 sensed using any available electrodes shown in FIG. 1A or in other examples described or shown in the accompanying drawings for processing and analysis according to the techniques disclosed herein.

Although IMD 110 of FIG. 1A is generally described herein as a pacemaker that delivers conduction system pacing and determines whether the pacing results in effective conduction system pacing, example systems including one or more implantable or external devices of any type configured to sense a cardiac EGM may be configured to implement the techniques of this disclosure.

In FIG. 1B, IMD 110 is an implantable cardioverter defibrillator device configured to receive internal defibrillator lead 124 extending through the RA and RV for delivering defibrillation pulses via elongate electrode 128 and sensing electrical signals via proximal electrodes 126, elongate electrode 128, and/or tip electrode 130. IMD 110 may be configured to sense intrinsic electrical signals of heart 102 of the patient and may be configured to detect one or more abnormal heart rhythms based on the intrinsic electrical signals of heart 102. IMD 110 may be configured to deliver one or more defibrillation pulses based on the detection of one or more abnormal heart rhythms to end the one or more abnormal heart rhythms and return the heart to normal, i.e., sinus, rhythm.

Defibrillator lead 124 may be advanced transvenously into the RV via the RA for positioning tip electrode 130 within inter-ventricular septum 104. Defibrillator lead 124 may be advanced further and/or a position of defibrillator lead 124 adjusted to position elongate electrode 128 proximate a wall of the RV. Elongate electrode 128 may be selected as defibrillation electrode (i.e., cathode) in combination with a can of IMD 110 as the return anode (i.e., anode). Proximal electrodes 126 may be ring electrodes or segmented ring electrodes. Proximal electrodes 126 may be configured to operate as sensing electrodes and/or pacing electrodes. Each proximal electrode of proximal electrodes 126 may be either a sensing or pacing electrode and may have concurrent and separate connection to IMD 110. Elongate electrode 128 may be a coil electrode. Elongate electrode 128 may be configured to operate as a sensing electrode, pacing electrode, and/or a defibrillation electrode. Tip electrode 130 may be secured into inter-ventricular septum 104. Tip electrode 130 may include any fixation feature as described herein. Tip electrode 130 may be configured to operate as sensing electrodes and/or pacing electrodes.

The cathode and the anode may be swapped. In some examples, the stimulation magnitude (e.g., pulse amplitude and/or pulse width) is selected to achieve suitable depolarization of heart 102 of the patient to end the arrhythmia. IMD 110 may be configured to thereafter deliver defibrillation one or more times if the therapy was unsuccessful. The therapy may be delivered at the same or higher stimulation magnitude. IMD 110 may additionally or alternatively be configured to thereafter sense intrinsic atrial P-waves and deliver atrial pacing pulses to set the heart rate of heart 102 of the patient.

FIG. 2 is a conceptual diagram that illustrates a distal portion of an example implantable medical lead 200 with a thread 212, e.g., a spacer thread. Lead 200 may include lead body 202, proximal electrode 204, spacer 206, and distal electrode 210 on distal end 208 of spacer 206. Spacer 206 may include thread 212 and may be described by spacer width 214. Thread 212 may be described by thread pitch 216 and thread height 218.

Lead 200 may be an implantable medical lead capable of implantation into a chamber of a heart, such as a right atrium or right ventricle of the heart. Rotation of lead 200 may engage thread 212 with cardiac tissue of the heart. A clinician may advance lead 200 into the cardiac tissue by rotating lead 200 until distal electrode 210 achieves a desired depth/position, e.g., for effective conduction system pacing. In some examples, lead 200 may an example of RA lead 116 of FIG. 1A or 1B. In some examples, lead 200 may an example of VCS pacing lead 118 of FIG. 1A or 1B. The cardiac tissue may cardiac tissue of any animal or human being.

Lead body 202 of lead 200 may have a proximal end e.g., proximal end 203 proximate a clinician and outside the vasculature during implantation, and a distal end, e.g., distal end 208. Although proximal end 203 is illustrated as a cylindrical end in FIG. 2, in some examples proximal end 203 may be a handle, an attachment point, a grip, or any other suitable attachment or connection to receive torque and/or contact from a clinician and/or a machine. Proximal end 203 may additionally be configured to electrically connect one or more electrodes, e.g., proximal electrode 204 or distal electrode 210, to an electrical source, e.g., IMD 110. Distal end 208 may be distal from proximal end 203 along longitudinal axis LA where longitudinal axis LA runs concentric with a center of lead 200. Distal end 208 may be connected to proximal end 203 via lead body 202 which may transfer rotational force from the clinician to distal end 208 of lead 200. A torque coil may be present within lead body 202 and may improve transfer of torque from the proximal end to the distal end. The torque coil may be electrical conductive. The torque coil may be electrical active. Distal end 208 may be configured to engage with cardiac tissue and deliver stimulation to the tissue of the heart.

Proximal electrode 204 may be a return electrode for stimulation delivered and/or a sensing electrode for sensing one or more signals from the implant location. Proximal electrode 204 may be referred to as a second electrode, where the distal electrode 210 is a first electrode. In some examples, such as those of FIG. 2, proximal electrode 204 is a ring electrode. Proximal electrode 204 may alternatively be any electrode or set of electrodes, such as two or more segmented electrodes (e.g., two or more radially displaced electrodes along the same plane transverse to the longitudinal axis of the lead) and/or two or more electrodes along longitudinal axis LA (e.g., proximal electrode includes a first proximal electrode and a second proximal electrode distal the first proximal electrode). Proximal electrode 204 may be used as an anode, e.g., by IMD 110. In some examples, proximal electrode 204 may be a stimulation electrode and may be used as cathode. Proximal electrode 204 may define a defibrillation electrode. Proximal electrode 204 may have a radial depth less than a radius defined by spacer width 214 (e.g., proximal electrode 204 is hollow along longitudinal axis LA) and as such a portion of lead body 202 may extend and continue underneath and longitudinally along proximal electrode 204. An electrical lead line (not shown) may electrically connect to proximal electrode 204 and run from proximal electrode 204, through lead body 202, to a connector (not shown) at proximal end 203 of lead body 202 that may be configured to connect to an IMD, such as IMD 110, at the proximal end of lead 200.

In some examples, proximal electrode 204 is optional and therefore lead 200 may not include proximal electrode 204. If proximal electrode 204 is omitted, spacer 206 may be continuous and/or contiguous with lead body 202 or spacer 206 may be omitted and lead body 202 may extend from a proximal end near a clinician to distal end 208. For example, lead body 202 may extend from proximal end to distal end may be continuous with and indistinguishable from spacer 206 except for one or more mechanical features at distal end 208 e.g., to support distal electrode 210.

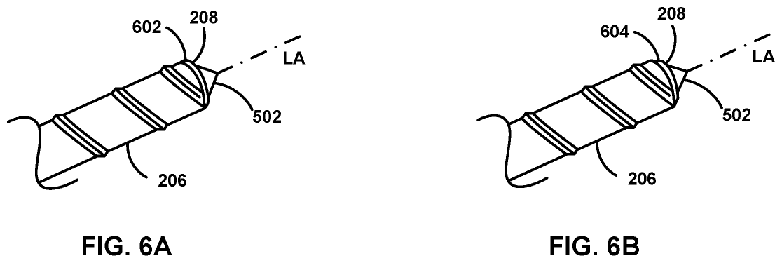

Spacer 206 may be a cylinder, or hollow cylinder, which defines a longitudinal distance, along longitudinal axis LA, between proximal electrode 204 and distal end 208. Spacer 206 may alternatively be described as a lead body and/or a tip to ring spacer. Spacer 206 may be formed of an electrically non-conductive and biocompatible material. A longitudinal length of spacer 206 may define a distance into tissue that the distal end of lead 200 may be inserted. For example, a longitudinal depth of spacer 206 may define a depth at which proximal electrode 204 begins to contact tissue of the heart, which may decrease a stimulation impedance and/or decrease measurement impedance. Distal end 208 of spacer 206 may be a flat circle as illustrated in FIG. 2. In some examples, distal end 208 or a cross section of spacer 206, may be an oval and therefore spacer 206 defines a first diameter in a first direction and a second diameter in a second direction. The first diameter may be different than the second diameter. Distal end 208 may take alternative shapes other than flat, such as those illustrated by FIG. 6A or 6B, e.g., chamfered or filleted, or may be otherwise suitably modified to increase penetration by distal end 208 into cardiac tissue.

Spacer width 214 may define a width of spacer 206 perpendicular to longitudinal axis LA. In some examples, spacer width 214 may be defined by a first diameter in a first direction and a second diameter in a second direction where the first direction may be orthogonal to the second direction and longitudinal axis LA. The first diameter may be different than the second diameter. Spacer width 214 may additionally or alternatively correspond to, e.g., be substantially equal to, a width of proximal electrode 204. In some examples, spacer width 214 may be wider than a width of lead body 202. In other examples, spacer width 214 may be equal to, or substantially equal to, the width of lead body 202. Spacer width 214 may be 4.1 French (Fr). Spacer width 214 may alternatively be 1 Fr, 2 Fr, 3 Fr, 4 Fr, 5 Fr, 6 Fr, 7 Fr, 8 Fr, 9 Fr, or any other suitable width capable of being inserted into tissue of the heart.

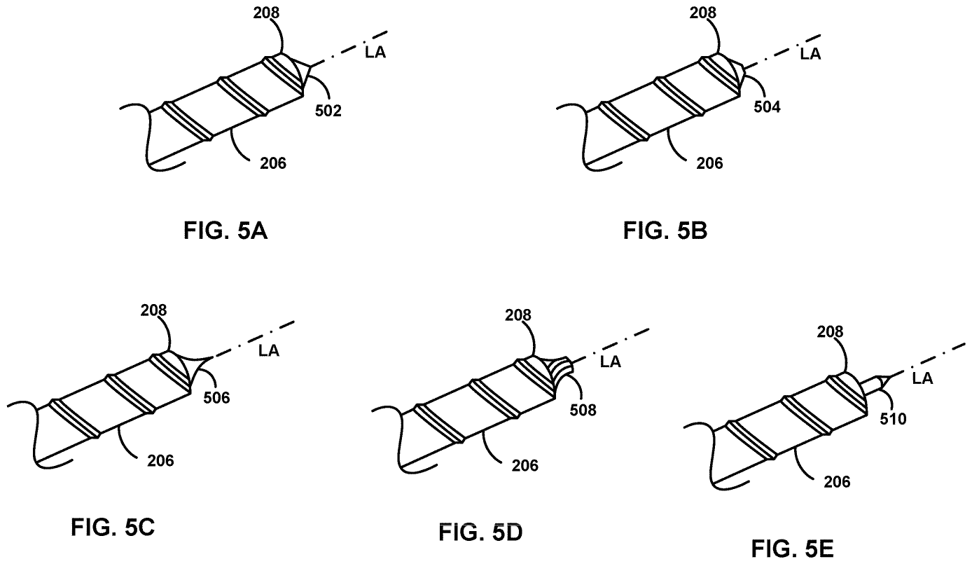

Distal electrode 210 may be an electrode within tissue configured to deliver stimulation to the tissue. Distal electrode 210 may be referred to as a first electrode, where proximal electrode 204 is a second electrode. In some examples, distal electrode 210 is a stimulation electrode and may be used as cathode. Distal electrode 210 may be used as an anode, e.g., by IMD 110. Distal electrode 210 may define a defibrillation electrode. In some examples, such as that of FIG. 2. distal electrode 210 is a helix. A helix electrode may have a pitch which defines a longitudinal depth the helix advances for each rotational turn. The helix electrode may provide advantages such improved control of implant depth for short depths, such as depths equal to or less than the depth of distal electrode 210. In some examples, the depth of distal electrode 210 may be 2 mm. In other examples, the depth of distal electrode 210 may be 0.5 mm, 1 mm, 1.5 mm, 2.5 mm, 3 mm, 3.5 mm, 4 mm, or any other suitable depth to stimulate tissue. Helix additionally may provide advantages such providing increased surface area over which to stimulate. Alternatively, distal electrode 210 may be any other suitable shape, such as those illustrated in FIGS. 5A, 5B, 5C, 5D, and/or 5E, where the advantages of such alternative shapes are discussed below. In some examples, distal electrode 210 may be segmented into a plurality (e.g., two or more) electrodes. For example, distal electrode 210 may be segmented such that a distal portion of distal electrode 210 defines a first electrode and a proximal portion of distal electrode 210 defines a second electrode electrically separate from the distal portion. In other examples, distal electrode 210 may be subdivided, i.e., segmented, into any number of electrically separate portions.

Distal electrode 210 may be a most distal point on lead 200 and therefore distal electrode 210 may be the first portion of lead 200 to contact with cardiac tissue when lead 200 is inserted into a body. During implantation, distal electrode 210 may be covered by a sheath until lead 200 reaches the heart of the body. Distal electrode 210 may be secured to distal end 208 of spacer 206 and/or lead body 202 through any method of attachment such as welding, integrally manufacturing, adhesives, or any other suitable attachment method to attach distal electrode 210 to distal end 208. Distal electrode 210 may be connected to an electrical lead line (not shown) which electrically connects to distal electrode 210 and runs from distal electrode 210, through spacer 206 if present, through and electrically disconnected from proximal electrode 204, and through lead body 202 to the proximal connector of lead 200 to connect to an IMD, such as IMD 110, at the proximal end of lead 200.

Thread 212 may define a thread on a radially outer surface of spacer 206, in the example illustrated by FIG. 2, or otherwise on a distal portion of lead body 202 proximal of distal electrode 210. Thread 212 may provide additional penetrating traction during implantation of lead 200 into tissue. An advantage of thread 212 and the additional penetrating traction may include improving implantation accuracy. For example, thread 212 may define pitch 216, where pitch 216 defines a depth along longitudinal axis LA lead 200 may advance into tissue per circumferential rotation of lead 200. In some examples, pitch 216 may be 0.0395 inches (in.) (i.e., 1 mm) per full rotational turn (i.e., rotation). In other examples, pitch 216 may be 0.1 mm/rotation, 0.25 mm/rotation (i.e., ~0.01 in./rotation), 0.5 mm/rotation, 0.75 mm/rotation, 1.25 mm/rotation, 1.5 mm/rotation, 1.75 mm/rotation, 2 mm/rotation, 2.5 mm/rotation (i.e., 0.1 in./rotation), 3 mm/rotation, or any other suitable depth per rotation and/or any value between the values listed above. As such, pitch 216 of thread 212 may improve an accuracy of implantation depth for lead 200 compared to distal electrode 210 alone because distal electrode 210 alone, when distal electrode 210 is defined by a helical electrode, only defines a pitch for the longitudinal depth of distal electrode 210.

Thread 212 may provide additional resistance against dislodgement. Specifically, thread 212, which is a simple machine (e.g., a screw) may resist longitudinal movement due to the ridges of thread 212. Thread 212 extends a radial height away from an outer surface of thread 212 as defined by height 218. In some examples, height 218 may be 8 or 10 thou, (i.e., 0.008 in (0.2 mm) or 0.010 (0.25 mm)). In other examples, height 218 may be 2.5 μm (i.e., 0.0025 mm or ~0.0001 in.), 0.025 mm, 0.05 mm, 0.075 mm, 0.1 mm, 0.15 mm, 0.175 mm, 0.225 mm, 0.275 mm, 0.3 mm, 0.35 mm, 0.5 mm, 0.75 mm, 1 mm, 1.25 mm, 1.5 mm, 1.75 mm, 2 mm, 2.25 mm, 2.5 mm (i.e., ~0.1 in.) or any other suitable height and/or any value between the values listed above. Specifically, height 218 defines the radial distance, i.e., transverse to longitudinal axis LA, away from an outer surface of spacer 206 that thread 212 extends. An increase in height 218 may increase a resistance to longitudinal movement of spacer 206 and lead 200 generally but may increase a volume of tissue affected by implantation. Thread 212 may be a bidirectional thread, i.e., thread 212 is smooth and lacks a one-way fixation device. Bidirectionally may provide the advantage of enabling removal and replacement by the clinician. Lead 200 is unlikely to be dislodged despite the bidirectionality because lead 200 may experience negligible rotational force once inserted into tissue and instead primarily experiences force along longitudinal axis LA which thread 212 is sufficiently able to resist.

Additionally, thread 212 may enable distal electrode 210 to take alternative shapes other a fixation helix, such as those illustrated in FIGS. 5A, 5B, 5C, 5D, and/or 5E, where the advantages of such alternative shapes are discussed below. For example, distal electrode 210 may be an electrode design other than a fixation helix such as a cone, a tapered cone, a dart, a frustum, or any other electrode designs as discussed throughout the disclosure below. As such, thread 212 on spacer 206 or otherwise on lead body 202 of lead 200 may provide the advantages such as improving lead placement (e.g., depth and location), improving cardiac pacing capture thresholds, reducing cardiac lead dislodgement, and enabling alternative distal electrode 210 designs which may thereby improve patient experiences and outcomes as well as improve physician implant experiences.

Thread 212 may be formed of the same material as spacer 206 and/or lead body 202. In some examples, thread 212 is formed of a different material than spacer 206 and/or lead body 202 such that thread 212 may be more pliable, malleable, or deformable than spacer 206 and/or lead body 202 to reduce the impact to tissue surrounding the implantation location. Thread 212 may be formed integrally with spacer 206 and/or lead body 202. In some examples, thread 212 is formed on the surface of spacer 206 and/or lead body 202 and connected to the surface of spacer 206 and/or lead body 202 through one or more suitable methods of fixation such as gluing, welding, or any other suitable method of affixing a first object to a second object where the first and second objects are both biocompatible.

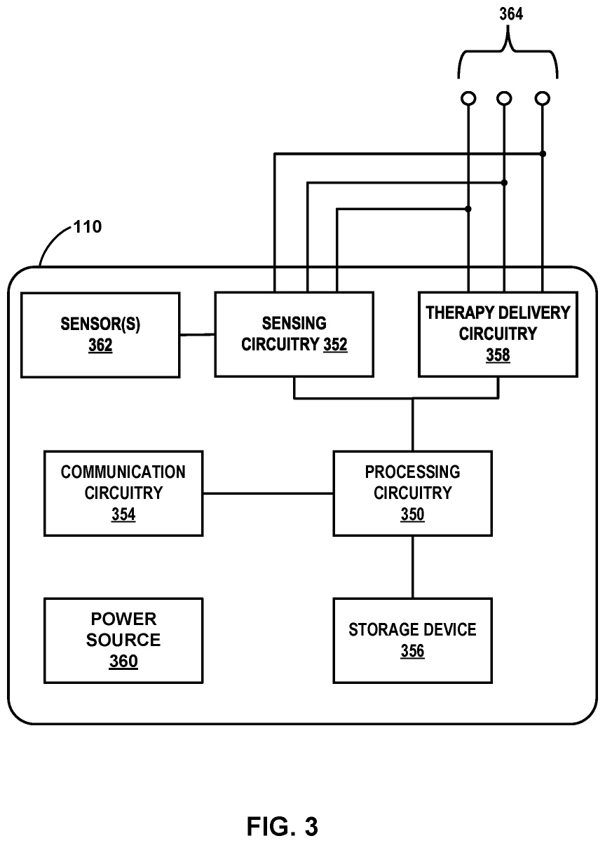

FIG. 3 is a functional block diagram illustrating an example configuration of IMD 110 of FIG. 1A or 1B in accordance with one or more techniques described herein. In the illustrated example, IMD 110 includes electrodes 364 which may be located on one or more leads (e.g., lead 200 of FIG. 2), processing circuitry 350, sensing circuitry 352, communication circuitry 354, storage device 356, therapy delivery circuitry 358, and sensors 362. Electrodes 364 may include electrodes described herein, such as proximal electrode 204 of FIG. 2 or distal electrode 210 of FIG. 2.

A housing of IMD 110 may enclose internal circuitry corresponding to various circuits and components for sensing cardiac signals from heart 102 of FIG. 1A or 1B and delivering cardiac pacing therapy including processing circuitry 350, sensing circuitry 352, communication circuitry 354, storage device 356, therapy delivery circuitry 358, and sensors 362. The housing may hermetically seal each of the one or more components within IMD 110 such as to keep out one or more fluids.

Processing circuitry 350 may include fixed function circuitry and/or programmable processing circuitry. Processing circuitry 350 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 350 may include multiple components, such as any combination of one or more microprocessors, one or more controllers, one or more DSPs, one or more ASICs, or one or more FPGAs, as well as other discrete or integrated logic circuitry. The functions attributed to processing circuitry 350 herein may be embodied as software, firmware, hardware or any combination thereof.

Sensing circuitry 352 may be coupled to electrodes 364. Sensing circuitry 352 may sense signals from electrodes 364, e.g., to produce a cardiac EGM, in order to facilitate monitoring the electrical activity of the heart. Sensing circuitry 352 also may monitor signals from sensors 362, which may include one or more accelerometers, pressure sensors, and/or optical sensors, as examples. In some examples, sensing circuitry 352 may include one or more filters and amplifiers for filtering and amplifying signals received from electrodes 364 and/or sensors 362. Sensing circuitry 352 may include switching circuitry for selecting electrodes 364 and their polarity for sensing the cardiac EGM signals described herein.

Sensing circuitry 352 and/or processing circuitry 350 may be configured to detect cardiac depolarizations (e.g., P-waves of atrial depolarizations, R-waves of ventricular depolarizations, etc.) when the cardiac EGM amplitude crosses a sensing threshold. For cardiac depolarization detection, sensing circuitry 352 may include a rectifier, filter, amplifier, comparator, and/or analog-to-digital converter, in some examples. In some examples, sensing circuitry 352 may output an indication to processing circuitry 350 in response to sensing of a cardiac depolarization. In this manner, processing circuitry 350 may receive detected cardiac depolarization indicators corresponding to the occurrence of detected R-waves and P-waves in the respective chambers of heart. Processing circuitry 350 may use the indications of detected R-waves and P-waves for determining inter-depolarization intervals, heart rate, and detecting arrhythmias, such as tachyarrhythmias and asystole.

Communication circuitry 354 may include any suitable hardware, firmware, software or any combination thereof for communicating with another device, such as external device 112, another networked computing device, or another IMD or sensor. Under the control of processing circuitry 350, communication circuitry 354 may receive downlink telemetry from, as well as send uplink telemetry to external device 112 or another device with the aid of an internal or external antenna. In addition, processing circuitry 350 may communicate with a networked computing device via an external device (e.g., external device 112) and a computer network, such as the Medtronic CareLink® Network. Communication circuitry 354 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, storage device 356 includes computer-readable instructions that, when executed by processing circuitry 350, cause IMD 110 and processing circuitry 350 to perform various functions attributed to IMD 110 and processing circuitry 350 herein. Storage device 356 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 356 may store, as examples, programmed values for one or more operational parameters of IMD 110 and/or data collected by IMD 110 for transmission to another device using communication circuitry 354. Data stored by storage device 356 and transmitted by communication circuitry 354 to one or more other devices may include digitized cardiac EGMs and other patient parameters.

Therapy delivery circuitry 358 may be configured to generate and deliver electrical therapy (e.g., pacing pulses) to heart 102 of a patient via electrodes 364. In some examples, therapy delivery circuitry 358 may include capacitors, current sources, and/or regulators. Therapy delivery circuitry 358 may include switching circuitry for selecting electrodes 364 and their polarity for delivering therapy signals to heart 102. Therapy delivery circuitry 358 may deliver electrical stimulation therapy via electrodes 364 according to one or more therapy parameter values, which may be stored in storage device 356. The therapy parameter values may, in the case of pacing pulses, include magnitude values, such as pulse amplitude and width.

IMD 110 may be configured to deliver LBBAP in order to provide synchronous LV contraction via selective or non-selective stimulation of the LBB or pacing on the left side of the ventricular septum, adjacent to the LBB. Asynchronous ventricle activation may be normalized or improved with the application of LBBAP. Patients with predominant left-sided conduction disturbances may achieve both intraventricular and interventricular synchrony if LBBAP is applied with proper timing such that paced LV activation fuses with native activation of the right ventricle. Achieving this result may involve suitable selection of the AV delay for LBBAP to match the patient's native AV conduction (e.g., as indicated by an interval between a P-wave and an intrinsic R-wave). For example, if a patient demonstrates a normal intrinsic AV conduction, properly timed LBBAP may provide AV synchrony as well as interventricular and intraventricular synchrony. Thus, if the patient's intrinsic AV conduction is normal (e.g., a P-R interval equal to or less than 200 milliseconds), cathodal capture of LBBAP may be preferred to AC pacing of the LBB area.

Processing circuitry 350 may be configured to control therapy delivery circuitry 358 to selectively deliver cathodal pacing and AC pacing based on patient parameters. In general, the patient parameters may indicate a health condition of a patient that may be more responsive to either cathodal pacing or bipolar stimulation. For example, patients with atrioventricular block, atrial fibrillation, etc., may benefit more from AC pacing than cathodal pacing. Thus, selectively delivering cathodal pacing and AC pacing based on patient parameters may result in better treatment, improving health outcomes.

Processing circuitry 350 may control therapy delivery circuitry 358 to deliver cathodal stimulation or AC stimulation based on whether a patient has prolonged atrioventricular conduction, also known as first-degree heart block or AV block Type I (or any other type of AV block, such as second-degree heart block, third-degree heart block, etc.). AV block is a cardiac conduction disorder characterized by an electrical signal transmission from the atria (upper chambers) to the ventricles (lower chambers) of the heart that is slower than normal, or completely blocked. In a healthy heart, the electrical impulses travel smoothly and rapidly through the AV node, which is responsible for transmitting signals between the atria and ventricles. For example, the P-R interval for a healthy heart may be 200 milliseconds or less.

In some examples, processing circuitry 350 may ascertain whether a patient has prolonged atrioventricular conduction based on whether a P-R interval of a patient satisfies (e.g., exceeds) a threshold (e.g., 200 milliseconds). If the P-R interval of a patient is equal to or less than the threshold, a patient may have a normal P-R interval, indicating that a patient has timely native activation of the right ventricle. Accordingly, fusing (e.g., blending or integrating) the cathodal pacing provided by lead 200 and the natural depolarization of the right ventricle may be desirable.

Processing circuitry 350 may measure the interval between an atrial sense (or an atrial pacing pulse) and ventricular sensing to measure the P-R interval. A normal or intrinsic P-R interval may be a P-R interval of 200 milliseconds or less. For atrial pacing, a normal P-R may be a P-R interval of about 250 milliseconds or less, assuming the atrial pacing rate is not excessively overdriving the normal sinus rate.

Therapy delivery circuitry 358 may deliver cathodal pacing using a tip electrode, such as distal electrode 210 as the cathode and a housing of IMD 110 as the ground. Alternatively, therapy delivery circuitry 358 may deliver cathodal pacing using a tip electrode such as distal electrode 210 as the cathode and a ring electrode, such as proximal electrode 204, as the ground, but at a magnitude that avoids anodal stimulation at proximal electrode 204. In this latter example, therapy delivery circuitry 358 may deliver cathodal stimulation by outputting the pulses at an amplitude equal to or less than 2 milliamps, though this amplitude may vary from patient to patient.

On the other hand, if the P-R interval of a patient is greater than the threshold, a patient may have prolonged atrioventricular conduction, indicating that a patient may no longer have timely native activation of the right ventricle. That is, if the patient's atrioventricular conduction is prolonged, hemodynamics suffer from the loss of AV synchrony and the detrimental effects of losing AV synchrony may outweigh the benefits of fusion between LBBAP and intrinsic right ventricular activation. In this situation, applying LBBAP earlier than optimal fusion between LBBAP and native right-sided activation may be suitable.

Processing circuitry 350 may control therapy delivery circuitry 358 to deliver AC pacing via distal electrode 210 and proximal electrode 204 to activate both ventricles of heart 102. In other words, distal electrode 210 may be selected as a pacing cathode electrode in combination with proximal electrode 204 as the return anode electrode for pacing and capturing the ventricles based on patient parameters (e.g., delayed or absent AV conduction, as indicated by a suprathreshold P-R interval). In other examples, proximal electrode 204 may be selected as a pacing cathode electrode in combination with distal electrode 210 as the return anode electrode for pacing and capturing the ventricles based on patient parameters. For example, processing circuitry 350 may configure the pacing magnitude of the pacing pulse delivered by therapy delivery circuitry 358. In some instances, the pacing pulse amplitude and/or pulse width may be selected to achieve only cathodal capture at the cathode electrode. In other instances, the pacing pulse amplitude and/or pulse width may be selected to achieve cathodal and anodal capture.

For example, processing circuitry 350 may cause therapy delivery circuitry 358 to deliver AC pacing by increasing the amplitude of the pulses (e.g., to 2.5 milliamps or greater), resulting in anodal stimulation by proximal electrode 204. The anodal stimulation may activate the right ventricle while the cathodal stimulation activates the left ventricle, correcting the long atrioventricular delay while providing interventricular and intraventricular synchrony in a patient with long AV delays.

Processing circuitry 350 may be configured to ascertain the proper amplitude for capture. For example, when pacing tip-to-can or tip-to-ring (e.g., distal electrode 210 to proximal electrode 204) (or in some examples tip-to-coil), processing circuitry 350 may ascertain the cathodal threshold for capture. In some examples, processing circuitry 350 may pace separately from ring to can or ring to coil to ascertain the capture threshold for anodal stimulation. In some examples, processing circuitry 350 may analyze changes in EGM morphology or the timing for depolarization to arrive at a distant sensing location as the pacing output changes. For example, processing circuitry 350 may pace tip-to-ring at low outputs. Processing circuitry 350 may use the EGM morphology as a baseline or reference for cathodal capture while increasing the pacing amplitude from tip-to-ring. Processing circuitry 350 may ascertain when the EGM morphology changes from the baseline (in response to an increase in the pacing amplitude), and in turn ascertain the threshold for A-C capture.

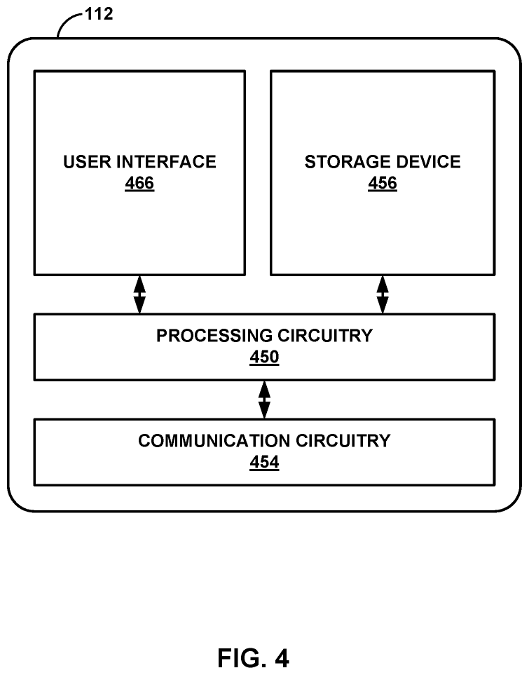

FIG. 4 is a block diagram illustrating an example configuration of components of external device 112. In the example of FIG. 4, external device 112 includes processing circuitry 450, communication circuitry 454, storage device 456, and user interface 466. External device 112 may be a computing device with a display viewable by the user and an interface for providing input to external device 112 (i.e., a user input mechanism). In some examples, external device 112 may be a notebook computer, tablet computer, workstation, one or more servers, cellular phone, personal digital assistant, or another suitable computing device that may run an application that enables the computing device to interact with IMD 110.

Processing circuitry 450 be analogous to processing circuitry 350 of FIG. 3. For example, processing circuitry 450 may include one or more processors that are configured to implement functionality and/or process instructions for execution within external device 112. For example, processing circuitry 450 may be capable of processing instructions stored in storage device 456. Processing circuitry 450 may include, for example, microprocessors, DSPs, ASICs, FPGAs, or equivalent discrete or integrated logic circuitry, or a combination of any of the foregoing devices or circuitry. Accordingly, processing circuitry 450 may include any suitable structure, whether in hardware, software, firmware, or any combination thereof, to perform the functions ascribed herein to processing circuitry 450.

Communication circuitry 454 be analogous to processing circuitry 350 of FIG. 3. For example, communication circuitry 454 may include any suitable hardware, firmware, software or any combination thereof for communicating with another device, such as IMD 110. Under the control of processing circuitry 450, communication circuitry 454 may receive downlink telemetry from, as well as send uplink telemetry to, IMD 110, or another device. Communication circuitry 454 may be configured to transmit or receive signals via inductive coupling, electromagnetic coupling, Near Field Communication (NFC), Radio Frequency (RF) communication, Bluetooth, WiFi, or other proprietary or non-proprietary wireless communication schemes. Communication circuitry 454 may also be configured to communicate with devices other than IMD 110 via any of a variety of forms of wired and/or wireless communication and/or network protocols.

Storage device 456 be analogous to processing circuitry 350 of FIG. 3. For example, storage device 456 may be configured to store information within external device 112 during operation. Storage device 456 may include a computer-readable storage medium or computer-readable storage device. In some examples, storage device 456 includes one or more of a short-term memory or a long-term memory. Storage device 456 may include, for example, RAM, DRAM, SRAM, magnetic discs, optical discs, flash memories, or forms of EPROM or EEPROM. In some examples, storage device 456 is used to store data indicative of instructions for execution by processing circuitry 450. Storage device 456 may be used by software or applications running on external device 112 to temporarily store information during program execution.

Data exchanged between external device 112 and IMD 110 may include operational parameters. External device 112 may transmit data including computer readable instructions which, when implemented by IMD 110, may control IMD 110 to change one or more operational parameters and/or export collected data. For example, processing circuitry 450 may transmit an instruction to IMD 110 which requests IMD 110 to export collected data (e.g., digitized cardiac EGMs) to external device 112. In turn, external device 112 may receive the collected data from IMD 110 and store the collected data in storage device 456.

A user, such as a clinician or a patient, may interact with external device 112 through user interface 466. User interface 466 includes a display (not shown), such as a liquid crystal display (LCD) or a light emitting diode (LED) display or other type of screen, with which processing circuitry 450 may present information related to external device 112 or IMD 110, e.g., cardiac EGMs, pacing magnitude, etc. In addition, user interface 466 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 450 of external device 112 and provide input. In other examples, user interface 466 also includes audio circuitry for providing audible notifications, instructions or other sounds to the user, receiving voice commands from the user, or both.

FIGS. 5A, 5B, 5C, 5D, and 5E (FIG. 5A-5E) illustrate example distal electrode configurations. FIG. 5A-5E are described with respect to lead 200 of FIG. 2 and more particularly with respect to distal end 208. Distal electrode 210 may be any distal electrode configuration such as cone electrode 502 of FIG. 5A, frustum electrode 504 of FIG. 5B, tapered cone electrode 506 of FIG. 5C, twisted frustum electrode 508 of FIG. 5D, and dart electrode 510 of FIG. 5E. Although described with respect to examples that include spacer 206, distal electrode configurations as described herein may be implemented on a distal end of a lead body 202 that does not include a spacer 206.

Each distal electrode configuration, e.g., electrodes 502-510, may be on distal end 208. Each distal electrode configuration may be an electrode inserted into tissue and configured to deliver stimulation to the tissue. The distal electrode configuration may be referred to as a first electrode. As described above with respect to FIG. 2., distal electrode configuration may be a helix. The distal electrode configuration may be a most distal point on lead 200 and therefore the distal electrode configuration may be the first portion of lead 200 to contact with cardiac tissue when lead 200 is inserted into a body. During implantation, the distal electrode configuration may be covered by a sheath until lead 200 reaches the heart of the body at which point the sheath may be removed and thereafter lead 200 may be inserted into cardiac tissue.

The distal electrode configuration may be secured to distal end 208, such as spacer 206 and/or lead body 202, through welding, integrally manufacturing, gluing, or any other suitable attachment method to attach the distal electrode configuration to distal end 208. The distal electrode configuration may be connected to an electrical lead line (not shown) which electrically connects from distal electrode configuration and runs underneath spacer 206 if present, under and electrically disconnected from proximal electrode 204, and through lead body 202 to connect to an IMD, such as IMD 110, at the proximal end of lead 200. The distal electrode configuration may be formed of any suitable electrode material capable of being biocompatible and delivering electrical stimulation to tissue.

The distal electrode configuration may be solid. In some examples, the distal electrode configuration is hollow. The distal electrode configuration may accommodate a guidewire or other implanted or nonimplanted feature passing therethrough. For example, the distal electrode configuration may accommodate a guidewire passing therethrough. In other examples, the distal electrode configuration may accommodate a stylet passing therethrough or being disposed therein. The guidewire and/or stylet may be electrically active. In other examples, the stylet or guidewire may be electrically inactive. A user may employ the guidewire and/or stylet may be for tracking, guidance, or navigation.

FIG. 5A represents a first example of the distal electrode configuration, cone electrode 502. Cone electrode 502 may be defined by an electrode shaped as a right cone, where a base of cone electrode 502 may be attached to or integral with distal end 208. Cone electrode 502 may be any geometric or non-geometric cones. In some examples, cone electrode 502 may be a non-right cone, such as an oblique cone. Cone electrode 502 may be rounded, tapered, and/or hemispherical. A vertex of cone electrode 502 may define the most distal point along longitudinal axis LA of lead 200. The vertex of cone electrode 502 may be the first point of contact between lead 200 and cardiac tissue when inserting lead 200 into cardiac tissue. Cone electrode 502 may taper at a constant rate between the base and the vertex of cone electrode 502. In some examples, the base of cone electrode 502 is equal in diameter to a diameter of spacer 206, represented by spacer width 214. The base of cone electrode 502 may be less than a diameter of spacer 206, represented by spacer width 214.

Cone electrode 502 may provide the advantage of reducing tissue harm compared to a blunt end of distal end 208 of spacer 206. For example, a gentle and linear slope between a point at the apex and the base at distal end 208 of spacer 206 may meticulously separate tissue. In contrast, the blunt end of distal end 208 of spacer 206, without a distal electrode configuration may impact tissue and separate the tissue in a crushing or tearing fashion, which may lead to more tissue damage than having any of the distal electrode configurations discussed herein. This damage may be localized and small in diameter, but an advantage of reducing damage at electrode implantation sites may include reducing stimulation thresholds due to reduced inflammation.

In an example, as cone electrode 502 is implanted in cardiac tissue, the vertex of cone electrode 502 impacts cardiac tissue first. The impact between the vertex of cone electrode 502 and the cardiac tissue creates an initial puncture. The clinician may advance cone electrode 502 into tissue by providing force along longitudinal axis LA. Cone electrode 502 may progressively widen an opening, through the use of a simple machine (e.g., a wedge) defined by the outer surface of cone electrode 502, to create an opening wide enough for the diameter of spacer 206, defined by spacer width 214. As such, cone electrode 502 may, through the use of simple machine (e.g., a wedge), provide the advantage of creating an initial puncture in the tissue in a manner which results in less tissue harm than compared to a separation of tissue with a blunt separator.

FIG. 5B represents a second example of the distal electrode configuration, frustoconical electrode 504. Frustoconical electrode 504 may be defined by an electrode shaped as a frustoconical, i.e., a cone with the longitudinally distal portion removed. A wide end of frustoconical electrode 504 may be attached to or integral with distal end 208. A narrow end of frustoconical electrode 504 may define the most distal point along longitudinal axis LA of lead 200. The narrow end of frustoconical electrode 504 may be the first point of contact between lead 200 and cardiac tissue when inserting lead 200 into cardiac tissue. Frustoconical electrode 504 may taper at a constant rate between the wide end and the narrow end. In some examples, frustoconical electrode 504 tapers at a changing rate between the wide end and the narrow end such that the surface connecting the wide end and the narrow end of frustoconical electrode 504 is curved. In some examples, the wide end of frustoconical electrode 504 is equal in diameter to a diameter of spacer 206, represented by spacer width 214. A diameter of the wide end frustoconical electrode 504 may be less than to a diameter of spacer 206, represented by spacer width 214.

Frustoconical electrode 504 may provide the advantage of reducing tissue harm compared to a blunt end of distal end 208 of spacer 206 alone. For example, a slope between the narrow end of frustoconical electrode 504 and the wide end of frustoconical electrode 504 at distal end may meticulously separate tissue. Narrow end of frustoconical electrode 504 may provide more blunt impact to the tissue than a point, such as those seen in cone electrode 502, tapered cone electrode 506, or dart electrode 510. However, the relatively more blunt end is significantly less blunt than distal end 208 of spacer 206, thereby reducing blunt force harm to the tissue, and additionally a relatively more blunt end may provide the advantage of decreasing non-purposeful (i.e., accidental) punctures. As frustoconical electrode 504 contacts and advances into tissue, frustoconical electrode 504 may progressively widen an opening, through the use of a simple machine (e.g., a wedge) defined by frustoconical electrode 504, to create an opening wide enough for the diameter of spacer 206, represented by spacer width 214. Frustoconical electrode 504, through the use of a simple machine (e.g., a wedge), may create the opening in a manner which results in less tissue harm than compared to a blunt separation of tissue.

FIG. 5C represents a third example of the distal electrode configuration tapered cone electrode 506. Tapered cone electrode 506 may be analogous in some aspects to cone electrode 502 and may deliver similar advantages. Tapered cone electrode 506 may be defined by an electrode shaped as a right cone, where a base of tapered cone electrode 506 may be attached to or integral with distal end 208. In some examples, tapered cone electrode 506 may be a non-right cone, such as an oblique cone. A vertex of tapered cone electrode 506 may define the most distal point along longitudinal axis LA of lead 200. The vertex of tapered cone electrode 506 may be the first point of contact between lead 200 and cardiac tissue when inserting lead 200 into cardiac tissue. Tapered cone electrode 506 may taper at a variable rate between the base and the vertex of tapered cone electrode 506. For example, and as shown in FIG. 5C, tapered cone electrode 506 may taper at an increased rate proximate the base of tapered cone electrode 506 but the tapering may decrease in rate proximate the apex of tapered cone electrode 506. Alternatively, tapered cone electrode 506 may taper at a decreased rate proximate the base of tapered cone electrode 506 but the tapering may increase in rate proximate the apex of tapered cone electrode 506. The base of tapered cone electrode 506 may be less than a diameter of spacer 206, represented by spacer width 214.

Tapered cone electrode 506 may provide the advantage of reducing tissue harm compared to a blunt end of distal end 208 of spacer 206. For example, a slope between a point at the apex and the base of tapered cone electrode 506 may meticulously separate tissue. In contrast, the blunt end of distal end 208 of spacer 206, without a distal electrode configuration, may impact tissue and separate the tissue in a crushing or tearing fashion, which may lead to more tissue damage than having any of the distal electrode configurations discussed herein. This damage may be localized and small in diameter, but an advantage of reducing damage at electrode implantation sites may include reducing stimulation thresholds through reduced inflammation.

In an example, as tapered cone electrode 506 is implanted, the vertex of tapered cone electrode 506 impacts tissue. The impact between tapered cone electrode 506 and cardiac tissue creates an initial puncture. A clinician may advance tapered cone electrode 506 into the cardiac tissue by providing force along longitudinal axis LA. Tapered cone electrode 506 may progressively widen an opening, through the use of a simple machine (e.g., a wedge) defined by the exponentially increasing circumference from the apex to the base of the outer surface of tapered cone electrode 506. This progressive widening may create an opening wide enough for the diameter of spacer 206, defined by spacer width 214. As such, tapered cone electrode 506 may, through the use of a simple machine (e.g., a wedge), provide the advantage of creating an initial puncture in the tissue in a manner which results in less tissue harm than compared to a separation of tissue with a blunter separator.

FIG. 5D represents a fourth example of the distal electrode configuration twisted frustum electrode 508. Twisted frustum electrode 508 may be defined by an electrode shaped as a frustum of a pyramid, i.e., a pyramid with the longitudinally distal portion removed, where a narrow end of the frustum is rotated around longitudinal axis LA with respect to the wide end of the frustum. The pyramid of twisted frustum electrode 508 may be a triangular pyramid, a square pyramid, a pentagonal pyramid, a hexagonal pyramid, an octagonal pyramid, or an N sided pyramid where N is an integer from three to one hundred. The N number of sides to the pyramid define the number of faces, the number of faces may define the number of edges, i.e., where two faces meet. The wide end of twisted frustum electrode 508 may be attached to or integral with distal end 208. The narrow end of twisted frustum electrode 508 may define the most distal point along longitudinal axis LA of lead 200. Due to the twisting of the frustum, the edges of the frustrum rotate around longitudinal axis LA.

The narrow end of twisted frustum electrode 508 may be the first point of contact between lead 200 and cardiac tissue when inserting lead 200 into cardiac tissue. Twisted frustum electrode 508 may taper at a constant rate between the wide end and the narrow end. In some examples, twisted frustum electrode 508 tapers at a changing rate between the wide end and the narrow end such that the surface connecting the wide end and the narrow end of twisted frustum electrode 508 is curved. In some examples, the wide end of twisted frustum electrode 508 is equal in diameter to a diameter of spacer 206, represented by spacer width 214. A diameter of the wide end of twisted frustum electrode 508 may be less than or equal to a diameter of spacer 206, represented by spacer width 214.

Twisted frustum electrode 508 may provide the advantage of reducing tissue harm compared to a blunt end of distal end 208 of spacer 206. For example, a slope between the narrow end of twisted frustum electrode 508 and the wide end of twisted frustum electrode 508 at distal end may meticulously separate tissue. Narrow end of twisted frustum electrode 508 may provide more blunt impact to the tissue than a point, such as those seen in cone electrode 502, tapered cone electrode 506, or dart electrode 510. However, the relatively more blunt end compared to cone electrode 502, tapered cone electrode 506, or dart electrode 510 is less blunt than distal end 208 of spacer 206 alone. As such, cone electrode 502 may reduce blunt force harm to the tissue, and additionally a relatively more blunt end, compared to cone electrode 502, tapered cone electrode 506, or dart electrode 510, may provide the advantage of decreasing accidental punctures. As twisted frustum electrode 508 contacts and advances into tissue, twisted frustum electrode 508 may progressively widen an opening, through the use of a simple machine (e.g., a wedge) defined by twisted frustum electrode 508, to create an opening wide enough for the diameter of spacer 206, represented by spacer width 214. Additionally, the twisted edges of twisted frustum electrode 508 may form cutting surfaces and/or additional wedge surfaces which may provide the added benefit of reducing blunt impact and decrease tissue disruption when separating tissue. Twisted frustum electrode 508, through the use of a simple machine (e.g., a wedge), may provide the advantage of creating the opening in a manner which results in less tissue harm than compared to a blunt separation of tissue and may provide the additional advantage of providing additional tissue separation surfaces along each edge of twisted frustum electrode 508.

FIG. 5E represents a fifth example of the distal electrode configuration dart electrode 510. Dart electrode 510 may be analogous in some aspects cone electrode 502 and may deliver similar advantages. Dart electrode 510 may be defined by an electrode shaped as a cone, where a base of the cone is attached to a cylindrical base, where the cylindrical base of dart electrode 510 may be attached to or integral with distal end 208. The cone of dart electrode 510 may be integrally formed with the cylindrical base of dart electrode 510. In some examples, the cylindrical base of dart electrode 510 may be welded, glued, or attached to distal end 208 in any other suitable attachment method. In other examples, dart electrode 510 may retract into distal end 208 and into spacer 206. Retracting dart electrode 510 into distal end 208 may include a wire pulling a portion of dart electrode 510 into spacer 206. Extending dart electrode 510 may include a wire pushing a portion of dart electrode 510 out from spacer 206. In other examples, the wire is a motor, a shaft, or any other suitable member capable of displacing dart electrode 510 at the command of a user. In some examples, the cone of dart electrode 510 may be welded, glued, or attached to the cylindrical base of dart electrode 510 in any other suitable attachment method.

The cone of dart electrode 510 may be a right cone. In some examples, the cone of the dart electrode 510 is a non-right cone, such as an oblique cone. A vertex of the cone of dart electrode 510 may define the most distal point along longitudinal axis LA of lead 200. The vertex of dart electrode 510 may be the first point of contact between lead 200 and cardiac tissue when inserting lead 200 into cardiac tissue. In some examples, dart electrode 510 may curve off longitudinal axis LA (i.e., an axis of dart electrode 510 may be non-colinear with longitudinal axis LA). Dart electrode 510 may be displaced off longitudinal axis LA at the control of a user. Alternatively, dart electrode 510 may be displaced off longitudinal axis LA by a preset deflection. In some examples, dart electrode 510 curves off of longitudinal axis LA by a set amount at a set rate. In other examples, the rate of curvature away from longitudinal axis LA changes (e.g., increases) with a distance from distal end 208.

Dart electrode 510 may taper at a variable rate between the base and the vertex of the cone of tapered dart electrode 510. In some examples, the cone of dart electrode 510 may taper at an increased rate proximate the base of the cone of dart electrode 510 but the tapering may decrease in rate proximate the apex of the cone of dart electrode 510. Alternatively, in some examples, the cone of dart electrode 510 may taper at a decreased rate proximate the base of the cone of dart electrode 510 but the tapering may increase in rate proximate the apex of the cone of dart electrode 510. The base of the cone of dart electrode 510 may be less than to a diameter of spacer 206, represented by spacer width 214. The cylindrical base of dart electrode 510 may have a constant diameter. In some examples, a cross section of the cylindrical base of dart electrode 510 is an oval such that the cylindrical base of dart electrode 510 defines a first diameter in a first direction and a second diameter in a second direction. The first diameter may be different than the second diameter. The diameter(s) of cylindrical base of dart electrode 510 may vary along the longitudinal axis LA.

Dart electrode 510 may provide the advantage of reducing tissue harm compared to a blunt end of distal end 208 of spacer 206. For example, a slope between a point at the apex and the base of the cone of dart electrode 510 may meticulously separate tissue. In contrast, the blunt end of distal end 208 of spacer 206, without a distal electrode configuration may impact tissue and separate the tissue in a crushing or tearing fashion, which may lead to more tissue damage than having any of the distal electrode configurations discussed herein. This damage may be localized and small in diameter, but an advantage of reducing damage at electrode implantation sites may include reducing stimulation thresholds due to reduced inflammation.

In an example, as dart electrode 510 is implanted, the vertex of the cone of dart electrode 510 impacts tissue. The impact between dart electrode 510 and cardiac tissue creates an initial puncture. The clinician may advance dart electrode 510 into the cardiac tissue by providing force along longitudinal axis LA. The cone of dart electrode 510 may progressively widen an opening, through the use of a simple machine (e.g., a wedge) defined by the outer surface of the cone of dart electrode 510. This widening may create an opening wide enough for the cylindrical base of dart electrode 510. The cylindrical base of dart electrode 510 may provide a stabilizing effect on the implantation location of the heart as the clinician applies force to push dart electrode 510 deeper into the tissue. As such, dart electrode 510 may, through the use of a simple machine (e.g., a wedge), provide the advantage of creating an initial puncture in the tissue in a manner which results in less tissue harm than compared to a separation of tissue with a blunter separator and may additionally, through the use of the cylindrical base of dart electrode 510, stabilize the location of implantation for dart electrode 510.

FIGS. 6A and 6B illustrate example spacers with modified distal ends. FIGS. 6A and 6B are described with respect to lead 200 of FIG. 2 and more particularly with respect to distal end 208 of spacer 206 and/or lead body 202, and distal electrode 210. A transition between spacer 206 and/or lead body 202 and distal end 208 may be a filleted edge 602 of FIG. 6A or chamfered edge 604 of FIG. 6B. The transition may be any other edge, such as a sharp edge as shown in FIGS. 2, 5A-5E and/or any other type of edge transitions suitable to reduce edge sharpness, reduce a bluntness of distal end 208, improve stress distribution, or improve manufacturability. In examples that do not include spacer 206, a distal end of lead body 202 may be similarly configured.

Spacer 206, as described above, is a cylinder or hollow cylinder which spaces distal electrode 210 from the proximal electrode 204. For example, without an edge modification, a flat end to a cylinder may define sharp edges such that face of distal end 208 is at 90 degrees relative to a surface of spacer 206. Sharp edges may result in increased force (i.e., pressure) applied to lead 200 to implant spacer 206 into tissue as the force is applied over a greater area. FIGS. 6A and 6B are illustrated with cone electrode 502 for the distal electrode; however, any distal electrode described herein may be substituted for cone electrode 502.

FIG. 6A represents a first example of an edge modification to the edge between spacer 206 and/or lead body 202 and distal end 208, where the edge modification is filleted edge 602. Filleted edge 602 applies an external fillet to the transition between spacer 206 and/or lead body 202 and distal end 208. An external fillet is a rounded corner on the edge between spacer 206 and/or lead body 202 and distal end 208. Filleted edge 602 may provide the advantages of reducing stress concentrations and improving manufacturability. Filleted edge 602 may additionally reduce a force of advancing lead 200 past distal end 208 into tissue until spacer 206 and/or lead body 202 contacts tissue because the surface area is reduced, thereby reducing a pressure, and additionally filleted edge 602 may act as a simple machine (e.g., a wedge) to improve implantability. Reducing a force of implantation may provide the advantage of reducing tissue damage to surrounding tissue.

FIG. 6B represents a second example of an edge modification to the edge between spacer 206 and/or lead body 202 and distal end 208, where the edge modification is chamfered edge 604. Chamfered edge 604 may be analogous in some aspects to filleted edge 602. Chamfered edge 604 applies an external chamfer to the transition between spacer 206 and/or lead body 202 and distal end 208. An external chamfer is a flattened corner on the edge between spacer 206 and/or lead body 202 and distal end 208. Chamfered edge 604 may provide the advantages of reducing stress concentrations and improving manufacturability. Chamfered edge 604 may additionally reduce a force of advancing lead 200 past distal end 208 into tissue until spacer 206 and/or lead body 202 contacts tissue because the surface area is reduced, thereby reducing a pressure. Chamfered edge 604 may additionally act as a simple machine (e.g., a wedge) to improve implantability. Reducing a force of implantation may provide the advantage of reducing tissue damage to surrounding tissue.

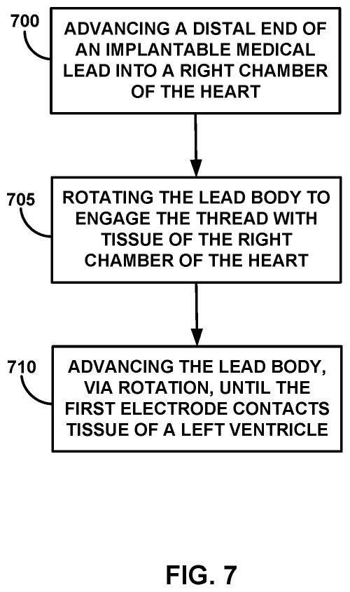

FIG. 7 is a flowchart of an example method for implanting a medical lead in accordance with techniques of this disclosure into a heart of a patient. The example method of implantation may apply to lead 200 with any modifications, (e.g., modifications to distal electrode 210 and/or distal end 208) as described throughout herein. The method of implantation may include advancing a distal end of an implantable medical lead into a right chamber of the heart (700), rotating the lead body to engage the thread with tissue of the right chamber of the heart (705), and advancing the lead body, via rotation, until the first electrode contacts tissue of a left ventricle (710). The example method of implantation may be utilized in different chambers, e.g., a right atrium, or other implant locations.

Advancing a distal end of an implantable medical lead into a right chamber of the heart (700) may include inserting an implantable medical lead, such as lead 200 of FIG. 2, into a peripheral vein. In some examples, lead 200 is inserted into a peripheral artery. A clinician may then advance, such as by applying force to lead body 202 of lead 200, distal end 208 of lead 200 through the peripheral vein into a right chamber of the heart, such as the right atrium or the right ventricle. The clinician may continue applying force to lead body 202 of lead 200 until distal electrode 210 achieves a desired depth/position, e.g., distal electrode 210 contacts the desired tissue of the right chamber of the heart for effective conduction system pacing. In some examples, if distal electrode 210 lacks a rotational pitch, such as cone electrode 502 of FIG. 5A, frustum electrode 504 of FIG. 5B, tapered cone electrode 506 of FIG. 5C, or dart electrode 510 of FIG. 5E then an additional application of force along longitudinal axis LA may be sufficient to puncture a portion of tissue of the right chamber of the heart and select a position of the right chamber of the heart for implantation. Alternatively, if distal electrode 210 defines a rotational pitch, such as helical electrode of FIG. 2 or twisted frustum electrode 508 of FIG. 5D, an additional application of force along longitudinal axis LA with a rotation about longitudinal axis LA may be sufficient to puncture a portion of tissue of the right chamber of the heart and select a position of the right chamber of the heart for implantation.

Once a position of the right chamber of the heart has been selected via an initial puncture, rotating the lead body may engage the thread with tissue of the right chamber of the heart (705). Rotating the lead body may include the clinician applying a rotational force about longitudinal axis LA which is transferred along lead body 202 of lead 200 which transfers the torque past or along proximal electrode 204 to spacer 206 (i.e., lead body). As spacer 206 and/or lead body 202 rotates, thread 212 may engage tissue of the right chamber of the heart. In some examples, where distal electrode 210 defines a rotational pitch, the rotational pitch of distal electrode 210 may match or be analogous to thread pitch 216 of distal electrode 210.

The example technique of FIG. 7 further includes advancing the lead body, via rotation, until the first electrode contacts tissue of a left ventricle and/or is proximate desired tissue for stimulation, e.g., conduction system pacing (710). Each full rotation of spacer 206 and/or lead body 202 after thread 212 has engaged with tissue of the right chamber of the heart, enables thread 212 to advance a distance defined by pitch 216. Specifically, pitch 216 defines a longitudinal distance between each thread of thread 212 which defines the distance that spacer 206 and/or lead body 202 may advance into tissue for each full rotation. As such, in step 715 a clinician may rotate lead body 202 a until proximal electrode 204 achieves a desired depth/position, e.g., distal electrode 204 contacts the desired tissue of the right chamber of the heart for effective conduction system pacing. The number of times the clinician rotates lead body 202 until proximal electrode 204 contacts achieves a desired depth is based on pitch 216 and a length along longitudinal axis LA of spacer 206 and/or lead body 202. For example, if the length along longitudinal axis LA of spacer 206 is X and the pitch is 0.1X/rotation, 10 or more full rotations may advance spacer 206 such that proximate electrode 204 contacts tissue.

In an example of steps 700-710, lead 200 may have dart electrode 510 and filleted edge 602. In step 700 a clinician may advance the dart electrode 510 and spacer 206 and/or lead body 202 into the right chamber of the heart as described above. In step 700 the clinician may additionally apply a force to insert dart electrode 510 into tissue of the right chamber of the heart. The clinician applying the force may additionally enable filleted edge 602 to advance into tissue of the right chamber of the heart because the pressure of insertion is reduced because the area of distal end 208 is reduced. In step 710, as force is applied by the clinician, thread 212 may engage with the tissue of the right chamber of the heart and rotation of spacer 206 and/or lead body 202 may enable thread 212 to engage with tissue of the right chamber of the heart. In step 715 each full rotation of spacer 206 and/or lead body 202 enables thread 212 to advance a distance defined by thread pitch 216. As such, in step 715 a clinician may rotate lead body 202 a defined number of times such that spacer 206 and/or lead body 202 advances a set distance into tissue of the right chamber of the heart such that proximal electrode 204 contacts tissue of the right chamber of the heart.

The following examples are illustrative of the techniques described herein.

Example 1. An implantable medical lead comprising: a lead body extending from a proximal end to a distal end along a longitudinal axis; a first electrode extending distally from the distal end; and a thread formed on a radially outer surface of the lead body proximal of the first electrode, the thread configured to engage tissue of a patient as the first electrode is advanced into the tissue.

Example 2. The implantable medical lead of example 1, further comprising a second electrode proximal of the thread.

Example 3. The implantable medical lead of example 2, wherein the lead body comprises a spacer connecting the first electrode to the second electrode, wherein the thread is formed on a radially outer surface of the spacer.

Example 4. The implantable medical lead of example 2 or 3, where the first electrode is a cathode and the second electrode is an anode.

Example 5. The implantable medical lead of any one or more of claims 2 to 4, wherein the second electrode comprises a ring electrode.

Example 6. The implantable medical lead of any one or more of examples 1 to 5, wherein the first electrode comprises a fixation helix coaxial with the longitudinal axis, the fixation helix comprising a circumferentially twisted wire.

Example 7. The implantable medical lead of example 6, wherein the fixation helix is a right-handed helix and the thread is a right-handed thread or wherein the fixation helix is a left-handed helix and the thread is a left-handed thread.

Example 8. The implantable medical lead of example 6 or 7, wherein the fixation helix has a pitch and the thread has the pitch.

Example 9. The implantable medical lead of any one or more of examples 1 to 5, wherein the first electrode comprises a cone, wherein a tip of the cone defines the distal end of the implantable medical lead.

Example 10. The implantable medical lead of any one or more of examples 1 to 5, wherein the first electrode comprises a frustoconical shape, wherein a narrow end of the frustoconical shape defines a distal end of the first electrode.

Example 11. The implantable medical lead of any one or more of examples 1 to 5, wherein the first electrode comprises a tapered cone, wherein a tip of the tapered cone defines a distal end of the first electrode.

Example 12. The implantable medical lead of any one or more of examples 1 to 5, wherein the first electrode comprises a twisted frustum, wherein a narrow end of the twisted frustum defines a distal end of the first electrode.

Example 13. The implantable medical lead of any one or more of examples 1 to 5, wherein the first electrode comprises a dart, wherein a narrow end of the twisted frustum defines a distal end of the first electrode.

Example 14. The implantable medical lead of any one or more of examples 1 to 13, further comprising a third electrode, wherein the third electrode is longitudinally between the first electrode and the second electrode along the longitudinal axis.

Example 15. The implantable medical lead of any one or more of examples 1 to 14, wherein a diameter of the lead body is within a range from 2 French to 6 French.

Example 16. The implantable medical lead of any one or more of examples 1 to 15, wherein a radial height of the thread is within a range from 0.001 inches to 0.1 inches and a pitch of the thread is within a range from 0.01 inches/turn to 0.1 inches/turn.

Example 17. The implantable medical lead of any one or more of examples 1 to 16, further comprising a torque coil connected to the lead body or the second electrode, wherein the torque coil is configured to transfer torque from a catheter to the lead body.

Example 18. The implantable medical lead of any one or more of examples 1 to 17, wherein the distal end of the lead body is chamfered.

Example 19. The implantable medical lead of any one or more of examples 1 to 18, wherein the distal end of the lead body is filleted.

Example 20. A method of implanting a medical device comprising: advancing a distal end of an implantable medical lead into a right chamber of the heart, wherein the implantable medical lead comprises: a lead body extending from a proximal end to a distal end along a longitudinal axis; a first electrode extending distally from the distal end; and a thread formed on a radially outer surface of the lead body proximal of the first electrode; rotating of the lead body, wherein the rotating is configured to engage the thread with cardiac tissue of the heart; and advancing the lead body via rotation until the first electrode contacts tissue of a heart's conduction system.

Example 21. The method of example 20, further comprising: connecting the cardiac stimulation lead to an implantable medical device configured to deliver conduction system pacing via the first electrode.

Example 22. The method of example 20 or 21, wherein the tissue of the heart comprises a Triangle of Koch, Bachman's bundle, or a ventricular septum of the heart.

Example 23. A medical device system comprising: an implantable medical lead comprising: a lead body extending from a proximal end to a distal end along a longitudinal axis; a first electrode extending distally from the distal end; and a thread formed on a radially outer surface of the lead body proximal of the first electrode, the thread configured to engage tissue of a patient as the first electrode is advanced into the tissue; and an implantable medical device comprising: processing circuitry configured to control stimulation circuitry, wherein the stimulation circuitry is configured to deliver cardiac stimulation via the first electrode to tissue of a left ventricle of a patient.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

What is claimed is:

1. An implantable medical lead comprising:
a lead body extending from a proximal end to a distal end along a longitudinal axis;
a first electrode extending distally from the distal end; and
a thread formed on a radially outer surface of the lead body proximal of the first electrode, the thread configured to engage tissue of a patient as the first electrode is advanced into the tissue.

2. The implantable medical lead of claim 1, further comprising a second electrode proximal of the thread.

3. The implantable medical lead of claim 2, wherein the lead body comprises a spacer connecting the first electrode to the second electrode, wherein the thread is formed on a radially outer surface of the spacer.

4. The implantable medical lead of claim 2, where the first electrode is a cathode and the second electrode is an anode.

5. The implantable medical lead of claim 2, wherein the second electrode comprises a ring electrode.

6. The implantable medical lead of claim 1, wherein the first electrode comprises a fixation helix coaxial with the longitudinal axis, the fixation helix comprising a circumferentially twisted wire.

7. The implantable medical lead of claim 6, wherein the fixation helix is a right-handed helix and the thread is a right-handed thread or wherein the fixation helix is a left-handed helix and the thread is a left-handed thread.

8. The implantable medical lead of claim 6, wherein the fixation helix has a pitch and the thread has the pitch.

9. The implantable medical lead of claim 1, wherein the first electrode comprises a cone, wherein a tip of the cone defines the distal end of the implantable medical lead.

10. The implantable medical lead of claim 1, wherein the first electrode comprises a frustoconical shape, wherein a narrow end of the frustoconical shape defines a distal end of the first electrode.

11. The implantable medical lead of claim 1, wherein the first electrode comprises a tapered cone, wherein a tip of the tapered cone defines a distal end of the first electrode.

12. The implantable medical lead of claim 1, wherein the first electrode comprises a twisted frustum, wherein a narrow end of the twisted frustum defines a distal end of the first electrode.

13. The implantable medical lead of claim 1, wherein the first electrode comprises a dart, wherein a narrow end of the twisted frustum defines a distal end of the first electrode.

14. The implantable medical lead of claim 1, further comprising a third electrode, wherein the third electrode is longitudinally between the first electrode and the second electrode along the longitudinal axis.

15. The implantable medical lead of claim 1, wherein a diameter of the lead body is within a range from 2 French to 6 French.

16. The implantable medical lead of claim 1, wherein a radial height of the thread is within a range from 0.001 inches to 0.1 inches and a pitch of the thread is within a range from 0.01 inches/turn to 0.1 inches/turn.

17. A method of implanting a medical device comprising:
advancing a distal end of an implantable medical lead into a right chamber of the heart, wherein the implantable medical lead comprises:
a lead body extending from a proximal end to a distal end along a longitudinal axis;
a first electrode extending distally from the distal end; and
a thread formed on a radially outer surface of the lead body proximal of the first electrode;
rotating of the lead body, wherein the rotating is configured to engage the thread with cardiac tissue of the heart; and
advancing the lead body via rotation until the first electrode contacts tissue of a heart's conduction system.

18. The method of claim 17, further comprising:
connecting the cardiac stimulation lead to an implantable medical device configured to deliver conduction system pacing via the first electrode.

19. The method of claim 17, wherein the tissue of the heart comprises a Triangle of Koch, Bachman's bundle, or a ventricular septum of the heart.

20. A medical device system comprising:
an implantable medical lead comprising:
a lead body extending from a proximal end to a distal end along a longitudinal axis;
a first electrode extending distally from the distal end; and
a thread formed on a radially outer surface of the lead body proximal of the first electrode, the thread configured to engage tissue of a patient as the first electrode is advanced into the tissue; and
an implantable medical device comprising:
processing circuitry configured to control stimulation circuitry, wherein the stimulation circuitry is configured to deliver cardiac stimulation via the first electrode to tissue of a left ventricle of a patient.
